(19) United States
(12) Patent Application Publication
(10) Pub. No.: US 2020/0147361 A1
(43) Pub. Date: May 14, 2020

(54) SKIN TREATMENT DEVICES AND METHODS

(71) Applicant: Mayo Foundation for Medical Education and Research, Rochester, MN (US)

(72) Inventors: Christopher V. DeSimone, Rochester, MN (US); Samuel J. Asirvatham, Rochester, MN (US)

(73) Assignee: Mayo Foundation for Medical Education and Research, Rochester, MN (US)

(21) Appl. No.: 16/610,659

(22) PCT Filed: Apr. 30, 2018
(86) PCT No.: PCT/US2018/030172

§ 371 Date: Nov. 4, 2019

Related U.S. Application Data
(60) Provisional application No. 62/500,871, filed on May 3, 2017.

(51) Int. Cl. A61N 1/04; A61B 5/01; A61B 5/00; A61B 5/053

(52) U.S. Cl. CPC A61N 1/0416; A61B 5/01; A61N 1/0468; A61B 5/0533; A61B 5/441

(57) ABSTRACT

Devices and methods can be used to treat dermatologic disorders using low dose DC electroporation. Such electroporation is essentially non-thermal modulation. In some implementations, the devices and methods described herein can be used for anti-aging treatments. In some implementations, the devices and methods described herein can be used for detecting/screening of various types of skin cancers. In some implementations, the devices and methods described herein can be used for therapy delivery for the treatment of early or pre-malignant skin cancers.

### CROSS-REFERENCE TO RELATED APPLICATIONS

This application claims the benefit of U.S. Provisional Application Ser. No. 62/500,871, filed May 3, 2017. The disclosure of the prior application is considered part of (and is incorporated by reference in) the disclosure of this application.

### BACKGROUND

### 1. Technical Field

This document relates to devices and methods for treating skin. For example, this document relates to devices and methods for treating dermatologic disorders using low dose DC electroporation.

### 2. Background Information

There are two primary factors that cause skin to age: 1) those due to the natural aging process and 2) secondary damage from the rays of the sun. Skin changes are among the most visible signs of aging. Evidence of increasing age includes wrinkles and sagging skin and striae or “stretch marks”.

Each year there are increasing numbers of new cases of skin cancer than the combined incidence of cancers of the breast, prostate, lung, and colon. The annual cost of treating skin cancers in the U.S. is estimated at $8.1 billion; about $4.8 billion for non-melanoma skin cancers and $3.3 billion for melanoma.

Skin cancer develops from uncontrolled growth of abnormal skin cells. It can occur when skin cell DNA is damaged and/or remains unrepaired (most often caused by ultraviolet radiation from sunrays or tanning beds). This can trigger mutations, or genetic defects in the DNA, that lead the skin cells to multiply rapidly and form malignant tumors. Skin cancers found and removed early can be curable. The types of skin cancers and pre-cancers include actinic keratosis, atypical moles, basal cell carcinoma, melanoma, Merkel cell carcinoma, and squamous cell carcinoma.

### SUMMARY

This document describes devices and methods for treating skin. For example, this document describes devices and methods for treating dermatologic disorders using low-dose DC electroporation. Such electroporation is a non-thermal modulation of skin. In some implementations, the devices and methods described herein can be used for anti-aging treatments. In some implementations, the devices and methods described herein can be used for screening of various types of skin cancers. In some implementations, the devices and methods described herein can be used for therapy delivery for the treatment of early or pre-malignant skin cancers. In some implementations, the devices and methods described herein can be used for treatment of acne. In some implementations, the devices and methods described herein can be used for skin sterilization and/or disinfection to prevent or treat infection. In some implementations, the devices and methods described herein can be used for treatment of striae or “stretch marks.” In some implementations, the devices and methods described herein can be used for promotion of skin/wound healing.

In one aspect, this disclosure is directed to a skin treatment device that includes: (i) a housing, (ii) a first electrode with at least a portion of the first electrode extending from the housing, (iii) a second electrode with at least a portion of the second electrode extending from the housing, (iv) an insulator coupled to the housing and interposed between the first electrode and the second electrode, (v) a camera coupled to the housing or the insulator, and (vi) a processor configured to control the skin treatment device to deliver DC electroporation energy from the first electrode in response to identifying an image captured by the camera as a skin irregularity. Such a skin treatment device may optionally include one or more of the following features. The skin treatment device may also include a skin temperature sensor. The skin treatment device may also include a humidity sensor. The skin treatment may also include an on-board power source for supplying the DC electroporation energy. The skin treatment device may also include a pair of galvanic skin resistance (GSR) electrodes for measuring conductance of skin. The skin treatment device may also include a skin-contact electrode embedded within the insulator or positioned on an opposite side of a skin-contacting side of the insulator.

In another aspect, this disclosure is directed to a method for treating a patient. The method for treating the patient includes positioning a skin treatment device in contact with skin of the patient. The skin treatment device includes: a housing; a first electrode, at least a portion of the first electrode extending from the housing; a second electrode, at least a portion of the second electrode extending from the housing; an insulator coupled to the housing and interposed between the first electrode and the second electrode; a camera coupled to the housing or the insulator; and a processor configured to control the skin treatment device. The method for treating the patient further includes delivering DC electroporation energy from the first electrode in response to identifying, by the processor, a first image captured by the camera as a skin irregularity.

Such a method for treating a patient may optionally include one or more of the following features. The skin irregularity may be a wrinkle. The skin irregularity may be a cancerous or pre-cancerous lesion. The skin irregularity may be a stretch mark or striae. The skin irregularity may be acne. The skin irregularity may be an open wound. The skin irregularity may be a partially healed wound. The skin irregularity may be an infected wound. The skin irregularity may be an infected wound with the presence of a bacterial biofilm. The DC electroporation energy may be delivered automatically in response to the processor identifying the image captured by the camera as the skin irregularity. The method may also include ceasing the DC electroporation energy delivery based on identification, by the processor in a second image captured by the camera, of a reddening of skin at or near the skin irregularity. The method may also include increasing an intensity of the DC electroporation energy delivery based on a humidity detected by a humidity sensor of the skin treatment device. The method may also include increasing an intensity of the DC electroporation energy delivery based on a skin conductance detected by a pair of galvanic skin resistance (GSR) electrodes of the skin treatment device. The delivering DC electroporation energy may include: emitting the DC electroporation energy from the first electrode; passing of the DC electroporation energy through the skin of the patient; and/or receiving of the DC electroporation energy by the second electrode.

Particular embodiments of the subject matter described in this document can be implemented to realize one or more of the following advantages. In some implementations, the devices and methods described herein can be utilized by a user in an in-home setting. Accordingly, the frequency of use and regularity of use can be enhanced in comparison to purely clinical or in the clinic treatments. Such increased regularity of use can be advantageously effective for increasing compliance, as well as enhancing chronic therapy delivery and early detection of skin cancers. In addition, in some implementations, the devices and methods described herein are configured with various sensors and logic control such that the safety and efficacy of the devices and methods are enhanced. In one such example, the devices can be equipped with a camera and the DC electroporation energy delivery can be automatically decreased or discontinued in response to a detection by the camera of increased redness of the skin (indicating early thermal injury or damage). In another example, the devices can be equipped with a sensor that detects sweat on the skin and the electroporation energy delivery can be automatically increased in response to detecting no decrease in the amount of sweat. Conversely, in some implementations plateauing of the decrease of sweat (i.e., a reduction in the rate of decrease of sweat) can be used to automatically decrease or cease electroporation energy delivery because when the sweat stops decreasing, this indicates that the desired effect of the electroporation energy has been achieved. Such automatic features can contribute to advantageous ease-of-use, thereby making home treatments practical, convenient, and effective.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a schematic diagram of an electroporation device for delivering dermatologic therapy in accordance with some embodiments provided herein.

FIG. 2 shows an example electroporation device that is used in conjunction with a smart phone for delivering dermatologic therapy.

FIG. 3 shows an example electroporation device that is used in conjunction with a facemask for delivering dermatologic therapy to facial areas.

FIG. 4 shows an example electroporation device that is used in conjunction with a dental appliance for delivering dermatologic therapy within the mouth.

Like reference numbers represent corresponding parts throughout.

### DETAILED DESCRIPTION

This document describes devices and methods for treating skin. For example, this document describes devices and methods for treating dermatologic disorders using low dose DC electroporation. Such electroporation is essentially a non-thermal mode of skin modulation. In some implementations, the devices and methods described herein can be used for anti-aging treatments. In some implementations, the devices and methods described herein can be used for detecting/screening for various types of skin cancers. In some implementations, the devices and methods described herein can be used for therapy delivery for the treatment of early or pre-malignant skin cancers. In some implementations, the devices and methods described herein can be used for therapy delivery to open skin wounds, infected skin wounds, healing skin wounds, and wounds that contain microbiologic biofilms. In some implementations, the devices and methods described herein can be used for therapy for acne. In some implementations, the devices and methods described herein can be used for therapy for striae or “stretch marks”.

Many similarities exist between botulinum toxin and low-grade DC electroporation delivered to cell membranes. Currently, botulinum toxin is used as BOTOX® via injection in the skin to temporarily paralyze the muscles that can cause furrowing of the overlying skin to treat wrinkles and other muscle-based skin damage. For example, BOTOX® injection into the corrugator superciliaris muscle is used to treat superficial frontal wrinkling. As an alternative, or in addition to such injections, this document discloses the use of a handheld device that can provide low-grade, frequent applications of DC electroporation energy to the skin and/or underlying muscles. The devices and methods provided herein also can be used to identify and treat early or pre-malignant skin cancers.

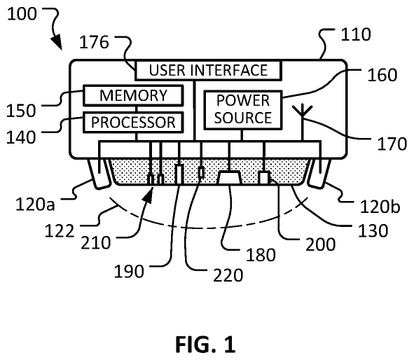

Referring to FIG. 1, an example device 100 can be used to treat dermatologic disorders using low dose DC electroporation. Device 100 includes a housing 110, a first electrode 120a, a second electrode 120b, and an insulator member 130. Device 100 can also optionally include one or more of components such as processors 140, memory 150, power source 160, an antenna 170, a user interface 176, a camera 180, a temperature sensor 190, humidity sensor 200, electrodes 210, and skin-contact electrode 220. Housing 110 provides the structure that the other components of device 100 are coupled to or contained within.

Housing 110 is represented schematically here. In actual practice, a number of different form factors of housing 110 are envisioned. In some implementations, housing 110 is a hand-held probe or module. In some implementations, housing 110 is curved such that electrodes 120a and 120b are non-parallel. Moreover, housing 110 can be flexible or malleable in some implementations. That is, in some implementations housing 110 can be selectively formed into a curved shape to facilitate use and efficacy of device 110.

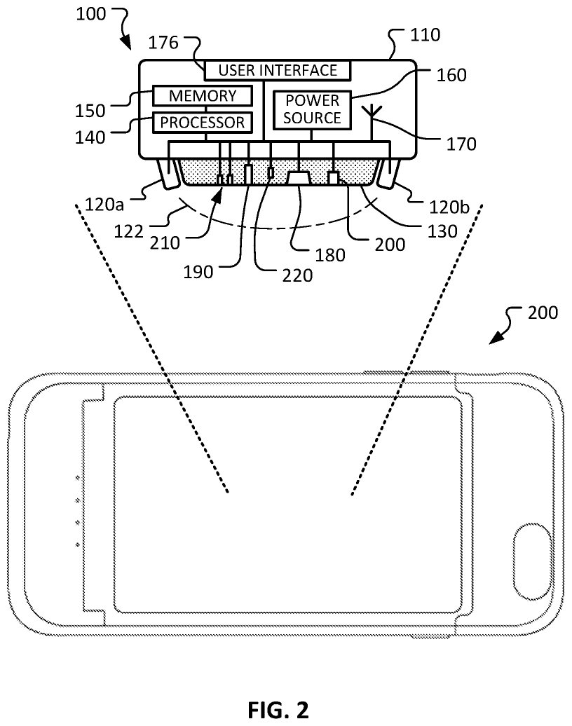

As depicted in FIG. 2, in some implementations, one or more aspects of device 100 are fully or partially implemented by a smart phone, and a cover with additional components of device 100 is coupled with the smart phone to comprise a skin treatment device 200. For example, in some implementations the smart phone's camera can comprise camera 180, the smart phone's battery can comprise power source 160, while the cover can include the electrodes 120a and 120b.

In some implementations, housing 110 can be attached to a handle so that the overall form is similar to that of a toothbrush, a hairbrush, a hand-held massager, or a back scratcher.

In some implementations, housing 110 can be embedded within clothing articles/garments. This can include the soles of shoes, sandals, or tennis shoes. This can include a strap to wrap around the chest, arm or arms, leg or legs, or head. This can include engagement of a belt or incorporated within a belt buckle.

In some implementations, housing 110 can include incorporation to dentures or dental retainer.

In some implementations, housing 110, can be attached to an enema-like device in order to treat rectal/anal skin cancers.

In some implementations, housing 110 his can also include attachment to a condom-like gadget to treat skin cancers that are resultant of sexually transmitted diseases, which can lead to skin cancers such as that caused by the Human Papilloma Virus (HPV).

In some implementations, housing 110 can be connected to a tampon-like device in order to treat vaginal or cervical skin cancers.

A small form (like a toothbrush size and shape, for example) can be especially useful for areas of the anatomy such as around the eyes, pubic regions, and in the mouth, to provide a few examples.

In some implementations, housing 110 is configured to be worn on the user's hand. For example, housing 110 can include one or more finger holes or channels. In some implementations, housing 110 is a glove. In some implementations, housing 110 is a watch. In some implementations, housing 110 is a watch. In some implementations, housing 110 is a wristband, armband, or headband. In some implementations, housing 110 is a knee-guard, ankle-guard, shin-guard, or mouth guard. In some implementations, housing 110 is an eye-patch.

Electrodes 120a and 120b are used to deliver low dose direct current (DC) electroporation to the user's skin to treat conditions such as, but not limited to, wrinkles, striae or “stretch marks”, acne, and various types of skin cancers. Accordingly, electrodes 120a and 120b are put into contact with the user's skin during treatment delivery.

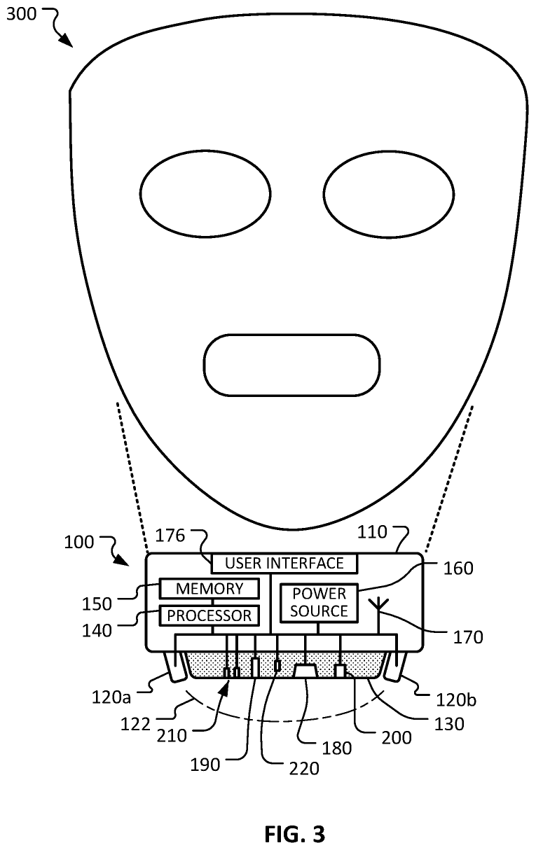

Referring to FIG. 3, as an example of a delivery method would be to connect the DC delivery unit and circuitry to a mask-like cover for uniform dispersal over the skin, such as one that could be worn over the face like a mask 300. However, this can be used over any skin surface and with a plurality of various configurations of skin patches that could be of various shapes and sizes and malleable in order to achieve site-specific contact for the patient. In some implementations, the low dose DC electroporation delivered by electrodes 120a and 120b is generally non-thermal. That is, in some cases the DC electroporation energy does not heat the skin appreciably. Electrodes 120a and 120b are a bipolar pair of electrodes. Accordingly, DC energy travels from a first one of electrodes 120a and 120b, through the user's skin, and is received by a second one of electrodes 120a and 120b (as depicted by line 122).

In some implementations, the longitudinal axes of electrodes 120a and 120b are not parallel to each other. Such an arrangement can help ensure that the DC energy delivered from one of electrodes 120a or 120b is received by the other of electrodes 120a or 120b. For example, in the depicted embodiment the longitudinal axes define an acute angle there between. In some implementations, the angle defined between the longitudinal axis of electrodes 120a and 120b is between about 0° and about 20°, or between about 10° and about 30°, or between about 20° and about 40°, or between about 30° and about 50°, or between about 40° and about 60°, or between about 50° and about 70°, or between about 60° and about 80°, or between about 70° and about 90°. In some implementations, the angle defined between the longitudinal axis of electrodes 120a and 120b is between about 90° and about 110°, or between about 100° and about 120°, or between about 110° and about 130°, or between about 120° and about 140°, or between about 130° and about 150°, or between about 140° and about 160°, or between about 150° and about 170°, or between about 160° and about 180°.

In some implementations, electrodes 120a and 120b are selectively extendable and retractable from housing 110. That is, in some implementations the user can manually extend and/or retract electrodes 120a and 120b in relation to housing 110. In particular implementations, electrodes 120a and 120b are automatically extendable and retractable from housing 110, for example in response to the beginning and ending of a therapy session. In some implementations, conductive elements (e.g., metallic particles) can be embedded within a sponge and the conductive elements can serve as electrodes 120a and 120b.

Device 100 also includes insulator 130. Insulator 130 is put into contact with the skin of the user during treatment delivery. Insulator 130 can be made of any appropriate material that is highly resistive to electrical conductance. In the depicted embodiment, insulator 130 is positioned between electrodes 120a and 120b. Accordingly, insulator 130 helps facilitate a current path between electrodes 120a and 120b that passes through the skin of the user. In some implementations, insulator 130 is configured like a sponge. The insulator can be used to provide localized and specific low dose DC electroporation treatment by facilitating the electric field to the skin on a certain area of the body.

Insulator 130 separates electrodes 120a and 120b. That separation distance can be various distances such as between about 1 mm to about 5 mm, or between about 4 mm to about 8 mm, or between about 7 mm to about 1.1 cm, or between about 1.0 cm to about 1.4 cm, or between about 1.3 cm to about 1.7 cm, or between about 1.6 cm to about 2.0 cm, or between about 1.9 cm to about 2.3 cm, or between about 2.2 cm to about 2.6 cm, or between about 2.5 cm to about 2.9 cm, or between about 2.8 cm to about 3.2 cm, or between about 3.0 cm to about 4.0 cm, or between about 3.8 cm to about 5.0 cm, or between about 4.8 cm to about 6.0 cm, or between about 5.8 cm to about 7.0 cm, or between about 6.8 cm to about 8.0 cm, or between about 7.8 cm to about 9.0 cm, or between about 8.8 cm to about 10.0 cm, or greater than 10.0 cm.

In the depicted embodiment, housing 110 of device 100 contains control circuitry including multiple modules, devices, circuits, and sub-systems that function cooperatively to perform the operations of device 100 as described herein. For example, the control circuitry in the housing 110 may include a combination of processor(s) 140 and the computer-readable memory 150 (which may optionally store executable instructions configured to cause the control circuitry to perform the sensing, determination, and therapy operations described herein).

Processor(s) 140 are suitable for the execution of one or more computer programs and include, by way of example, both general and special purpose microprocessors, and any one or more processors of any kind of digital computer. Processor(s) 140 can execute instructions, including the executable instructions that are stored in memory 150. In some implementations, processor(s) 140 may be implemented as a chipset of chips that include separate and multiple analog and digital processors. Processor(s) 140 may provide, for example, for coordination of the other components of device 110, such as control of delivery of electroporation energy from electrodes 120a and 120b, applications run by device 100, and wireless communications via a communication module and antenna 170.

Computer-readable memory 150 stores information within device 100, including, but not limited to, the executable instructions that can be used by processor(s) 140. Memory 150 can be implemented as one or more of a computer-readable medium or media, a volatile memory unit or units, or a non-volatile memory unit or units. An expansion memory may also be provided and connected to device 100 which may include, for example, a SIMM (Single In-Line Memory Module) card interface. The expansion memory may provide extra storage space for device 100, or may also store applications or other information for the device 100. Memory 150 may include, for example, flash memory and/or NVRAM memory (non-volatile random access memory).

Executable instructions can be stored in memory 150, the expansion memory, memory in processor 140, or in a combination thereof. The executable instructions can include instructions that, when executed, perform functions related to the operating systems of device 100 (e.g., operations of the user interface, coordination of intra-device module communications, control of the delivery of the DC electroporation energy from electrodes 120a and 120b, coordination and control of applications run by device 100, and so on). In addition, in this embodiment the executable instructions include instructions that, when executed, perform one or more of the functions and methods described elsewhere herein in relation to physiological parameter monitoring, analysis of the monitored parametric data, alarming, and communications with other devices and systems, as well as clinicians or monitored technicians. In some implementations, the executable instructions, or portions thereof, can be received in a propagated signal, for example, via the communication modules and antenna 170.

In some implementations, device 100 also includes an on-board power source 160. Power source 160 can provide the electroporation energy and the energy to operate the other devices and systems of device 100. In some embodiments, power source 160 includes one or more AC batteries and includes an AC/DC converter/rectifier. In some embodiments, power source 160 includes one or more batteries such as a non-rechargeable alkaline battery. In some embodiments, power source 160 includes one or more rechargeable batteries such as a nickel-metal hydride, lithium ion, lithium polymer, or zinc oxide battery. In particular embodiments, a combination of the aforementioned types of batteries are used, and a combination of rechargeable and non-rechargeable batteries can be used. The rechargeable batteries may be recharged by electrically coupling an external power source to the battery, or to a battery charging circuit in housing 110 that is electrically connected to power source 160. In some embodiments, the coupling of the external power source to device 100 is via a wired connection, such as by plugging a cord into a receptacle located on housing 110. The coupling may also be accomplished in some embodiments by the use of a docking station with which device 100 can mate to establish an electrical connection. In particular embodiments, the electrical coupling can be accomplished inductively (wirelessly). That is, an electrical coil that is within housing 110 can be wired to a battery charging circuit in housing 110. The internal electrical coil can receive inductive energy via an alternating magnetic field emanating from a primary coil that is part of an external charging station. An alternating current is thereby induced in and transmitted from the internal coil to the battery charging circuit in housing 110. The battery charging circuit can rectify the alternating current to produce direct current that is used to charge power source 160. In some implementations, device 100 receives power for operations via a wired connection with a power source. In some implementations, device 100 receives power for operations via a wired connection with a power source in a central hub such as a clinician's office or a provider's office.

In some implementations, device 100 can wirelessly communicate with other separate computerized devices and/or networks. As such, in some implementations device 100 includes a communication module and antenna 170. For example, in some embodiments device 100 can house one or more wireless communication devices configured to communicate using short-range wireless communication modes. Examples of such short-range communication modes that can be utilized by device 100 can include, but are not limited to, infrared (IR), radio frequency (RF), Wi-Fi, Bluetooth, ANT+, radio-frequency identification (RFID), near-field communications (NFC), IEEE 802.15.4, and IEEE 802.22. In addition, in some embodiments device 100 can house one or more wireless communication devices configured to communicate with some of the external devices using various types of long-range wireless modes. Examples of such long-range communication modes implemented by the sensing device 100 can include, but are not limited to, cellular communications, network communications (e.g., internet, intranet, telephone networks, broadband phone service, broadband networks, wide area networks, and local area networks).

To provide for interactions with a user, device 100 can also include a user interface 176. User interface 176 includes devices and systems to receive inputs to device 100, and to provide outputs from device 100. For example, in some embodiments user interface 176 can include a display (in some embodiments the display is a touchscreen display), one or more buttons that can be soft keys or hard keys, one or more audio speakers, one or more lights, a microphone, a camera, tactile feedback mechanisms (e.g., vibratory alarm signals), and the like. Using such devices, user interface 176 can receive user input including voice input, touchscreen input, soft key inputs, and the like. User interface 176 can also provide outputs including audible alarms or messages, visual alarms or messages, tactile alarms or messages, differentiation of alarm types, and the like.

In some implementations, device 100 can also optionally include one or more accelerometers mounted within housing 110, and/or other types of motion sensors such as gyroscopes. The accelerometers are electronic components that measures tilt and motion. The accelerometers are also capable of detecting rotation and motion gestures such as swinging or shaking. The accelerometers can be used to detect motion, and/or a lack of motion, which can then be communicated to processor 140 as an input signal and used in algorithms being run by processor 140. In some embodiments, the accelerometers can be configured to detect gestures for particular input commands, such as the acknowledgement of an alarm and other types of commands.

In some implementations, device 100 also includes camera 180. Some implementations include two or more cameras 180. Camera 180 can be used to take digital photos and/or video of the user's skin. Camera 180 can be used in conjunction with an image template matching program (e.g., run by processor 140 and memory 150) to identify normal skin and/or skin abnormalities such as, but not limited to, wrinkles, discolorations, skin cancers, acne, striae or “stretch marks”, and other abnormalities. When such abnormalities are detected, in some implementations low dose DC electroporation energy can be automatically delivered to the abnormality. In some implementations, one or more light sources are included as part of device 100 to enhance the image quality produced by camera 180. In some implementations, processor 140 is configured to perform image processing as well as image matching operations.

Device 100 also includes temperature sensor 190. Temperature sensor 190 can be used, for example, to detect an increase in skin surface temperature that can signify a warming of the skin in response to the DC electroporation energy. The signal from temperature sensor 190 can be received by processor 140 which can use the temperature signal as an input to various logical algorithms, including one for the discontinuation in DC electroporation energy delivery in response to a skin temperature increase above a particular threshold value.

In some embodiments, temperature sensor 190 can include a thermistor that is used to detect the skin surface temperature, and the thermistor can be mounted to insulator 130 and at least partially exposed along the surface of insulator 130. A thermistor is a type of resistor whose resistance varies significantly with temperature, more so than standard resistors do. In some embodiments, the thermistor is within a bridge circuit of temperature sensor 190. Temperature sensor 190 can periodically measure or continuously measure the skin surface temperature. The measured skin temperature value, and/or trends over time of such values, can be used to detect a change in skin temperature that may be determined to be at or above a threshold level that is indicative of excessive skin warming in some cases. In addition, temperature sensor 190 can be used to confirm that the user is operating device 100 properly in contact with the user's skin. That is, in some implementations device 100 can be configured to send a corresponding message via user interface 176 indicating that skin contact should be increased is an expected skin temperature is not detected during use.

In some implementations, device 100 can also include humidity sensor 200. Humidity sensor 200 can be used to provide an indication of the sweat of the user's skin. Such information can be useful for controlling the operations of device 100 because electroporation can, and is expected to cause sweat ducts to become temporarily impaired or dysfunctional. Therefore, for example, if a decrease in humidity is not detected by humidity sensor 200, in response, electroporation energy delivery can be increased in intensity or duration until an expected decrease in humidity is detected by humidity sensor 200. Further, if humidity does decrease as expected/desired, an end of the decreasing trend can be used as a signal to cease delivery of the electroporation energy in that area of the skin.

In some implementations, device 100 can also include electrodes 210.

Electrodes 210 can be used to measure intra-dermal conductance or the galvanic skin resistance (GSR) of the user's skin. GSR refers to the measured electrical resistance between two electrodes when a very weak current is steadily or periodically passed between them. Accordingly, some embodiments of electrodes 210 include two electrodes that are in contact with the skin of the user. For example, the two electrodes can be mounted on or within insulator member 130 in some implementations. Electrodes 210 can be spaced apart from each other and at least partially exposed so as to make contact with the skin. Device 100 can periodically measure the resistance between the two electrodes 210. The measurement, and/or a trending of multiple measurements over time can be used as an input to an algorithm for detecting the sweat of the skin. In some implementations, the signals from electrodes 210 and humidity sensor 200 are used in combination, to cross-check each other, for example.

Device 100 can use electrodes 210 as an electrical resistance-type moisture sensor that utilizes the relationship between the amount of moisture on the skin and the electrical resistance of the skin. In particular, electrodes 210 can operate on the principle that skin's resistance to the flow of electricity is lessened with increasing amounts of moisture such as perspiration. When the skin's GSR is lessened by perspiration, it can more readily conduct electricity and the flow of electricity can be detected by a monitoring circuit of device 100. Using these principles, electrodes 210 can be used to detect the presence of perspiration that may be determined to be at or above a threshold level amount of perspiration that is indicative of the delivery and efficacy of electroporation energy.

In some implementations, electrodes 210 can also be used to directly measure dermal and neural activity/inactivity (e.g., electrogram-based neural recording, electromyography or EMG for short). Such measurements can be useful for identifying the presence of skin lesions because the normal neural skin activity is tremendously impaired with all three common skin cancers (basal cell carcinoma, melanoma, and squamous cell carcinoma), and therefore detection of abnormal neural skin activity is an indicator of early skin tumors. Moreover, normal sweat activity is impaired with all three common skin cancers. For example, melanoma cells do not have sweat glands and also do not have the normal neural networking. Therefore, melanoma looks and behaves differently than normal healthy skin, such that the use of humidity sensing, electrogram-based neural recording, intra-dermal conductance, and imaging can be utilized to detect skin cancer by device 100.

In some implementations, device 100 can also include skin-contact electrode 220. A portion of insulator 130 can be interposed between skin-contact electrode 220 and the skin-contacting outer surface of insulator 130. Accordingly, a capacitance is created between skin-contact electrode 220 and the skin-contacting outer surface of insulator 130. That capacitance is affected by whether the skin-contacting outer surface of insulator 130 is actually in contact with the user's skin or not. Accordingly, skin-contact electrode 220 can provide an input signal to processor 140 that is indicative of whether insulator 130 is in contact with the user's skin or not. In some implementations, two or more skin-contact electrodes 220 are included in device 100.

Moreover, as a result of the electroporation delivery, a change to the skin's electrical properties is expected. Therefore, skin-contact electrode 220 can be used to detect such changes. Such signals from skin-contact electrode 220 can be provided to processor 140 which can use the signals to access whether changes (e.g., intensity increases, intensity decreases, cessation, etc.) to the delivery of electroporation energy should be made.

### Additional Optional Features

In some implementations, feedback and control of device 100 is accomplished at least in part by arranging small electrodes with a very small bipiole (e.g., <1 mm spacing) along with larger spaced electrodes in order to allow for noise cancellation. At the same time, a myopotential can be recorded and electroporation is only applied until a slowing of intrinsic myopotential activity occurs, without fragmentation (no skin burning).

In some implementations, feedback and control of device 100 is accomplished at least in part by the use of temperature sensor 190 so that electroporation automatically shuts off whenever a thermal or injury is being generated and detected.

In some implementations, feedback and control of device 100 is accomplished at least in part by integrating this device in a cell phone cover that can be integrated with the circuitry of a smart phone/cell phone. In this innovation, the battery from the smart phone is used to power the device and recharged as usual to do this. The novelty arises from the use of the camera capability of the cell phone to serve as another feedback circuit by monitoring skin tone, skin color, and skin tonography. This may also involve the individual keeping a pictorial diary with a form of visual recognition software to compare for signs of improvement, worsening, or no effect over time in an automated and objective manner. In some implementations, clamp-like electrodes that not only are folded back into the cell phone case but can come out and then be placed like a band around either the arm, leg, or when expanded, head, chest, are included. In addition, in some implementations electrolytic gels would be placed within the case so as to serve to diffuse both the anode or cathode to varying extents to create a manipulatable field, which would be an important adjunct to simply varying the current intensity. This also provides a method to treat the skin disorders in a longitudinal manner as they spread or shrink, and thus the electrode coverage and electroporation area can be varied as desired.

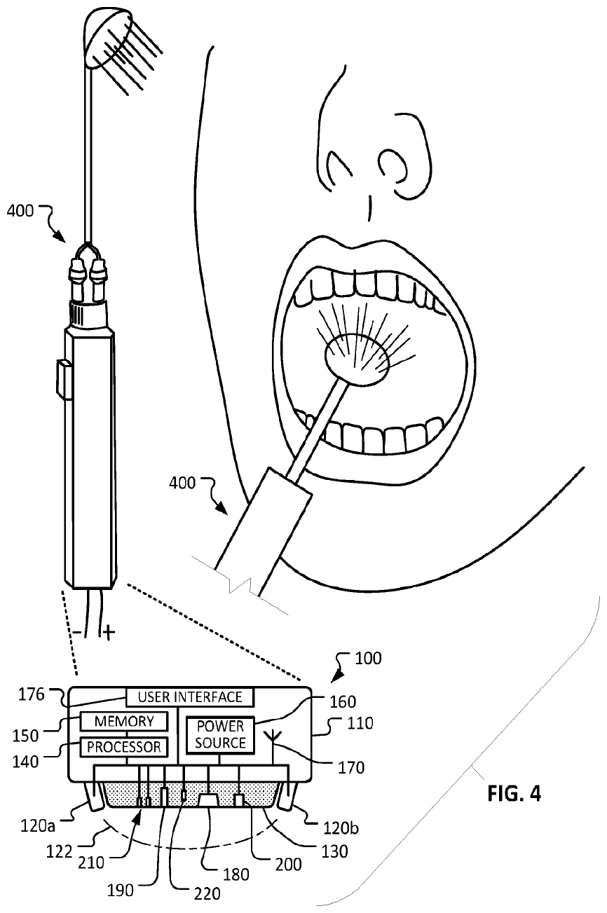

Referring to FIG. 4, an additional application for such a handheld device as well as the above-mentioned monitoring, feedback, and control techniques can be applied to prevent early malignancy via a device for teeth cleaning 400 that can deliver low-dose DC electroporation. As faster dividing, malignant cells are more prone to the effects of electroporation, device 400 can deliver low-dose DC electroporation to the teeth, gums, buccal mucosa, and local skin in order to prevent malignant tendencies in these cells. Similarly, the electroporation device 400 can in some cases provide a way of cleansing oral mucosa from those at risk for carcinogen induced changes such as in chronic smokers or tobacco chewers. Similarly, the electroporation device 400 may also serve to as a way to prevent the formation of microbiologic biofilms on teeth. The devices and methods described herein may also be suitable for delivering DC energy to breakdown biofilms that can form on teeth.

For example, daily use of a toothbrush configured to deliver low-dose electroporation may serve to provide a means to remove, prevent, or eliminate biofilm formation or degrade biofilms on the teeth. The differential components of microbiologic biofilms versus normal teeth, gums, and enamel can provide for prevention of infection that could lead to bacterial endocarditis. This can be especially useful in patients with indwelling medical instruments such as valves, tissue grafts, artificial knees, cardiac conduits, cardiac transplants, as a means of prevention of bacterial valvular endocarditis by eliminating this common source of mouth flora that can lead to such diseases. This can be performed on an as needed basis or in a daily fashion for long-term prevention in certain individuals. Similarly, this can be used as a method to treat dental caries by accelerated the destruction of abnormal cells.

Such a tool as the electroporation device 400 may also be of utility during dental work and manipulation. For example, one embodiment would include the above-mentioned DC delivery unit connected to a tooth watering pick. This could be used during dental work in order to not only function for teeth cleaning and sterilization, but also in order to provide control of bleeding, as well as analgesia, via a virtual electrode effect with an electrolyte based solution such as but not limited to saline irrigation applied to the area of dental work (refer again to FIG. 4). Such a device 400 could be constructed to serve as an at-home device for use in a scheduled manner for chronic therapy for chronic suppression and for optimum hygiene. In addition, these version of the device 400 can also be used to prevent or treat gum disease, pre-cancerous or cancerous oral lesions such as that of the buccal mucosa, gums, and tongue. The use of an irrigating solution, such as that of normal saline could provide a titratable aspect for which to create a virtual electrode effect of low-dose DC energy delivery.

In some implementations, the use of feedback sensors including electrocutaneous potentials, high resolution photography from the device maintaining the electrical field, sweat characteristics, and optical coherence tomography in lieu of high resolution microscopy to understand when electroporation energy should be stopped, and for patients to have self-surveillance (much like self-examination of other organs to monitor for early malignancy) or for the right time for a second or subsequent therapy at a particular sight for aging effects, is facilitated and provided using device 100.

Inter-dermal electrodes much like our subcutaneous recording electrodes could serve as the return limb for the current, and the direct current itself could be used as an anesthetic. That could be particularly useful for high-risk lesions. This could also be useful for open lesions, especially where erosion of skin integrity occurs. This could provide adequate analgesia of the surrounding skin with intact nerve fibers. The bipolar design of device 100 is advantageous. A lot of the existing devices/systems surprisingly use a single electrode surface that presumably will require a return patch at an unspecified site. The inventors know from experimental data that this is not a good idea because of undirected current flow and potential muscle twitching.

Some methods to have a bipolar design can include, but are not limited to: for the smartphone approach, the case can serve as the anode/cathode and the phone transmitting app can serve as the cathode/anode; a standalone device can include a mesh that may or may not be bioabsorbable and placed on the skin under the electroporation emitting device (since the mesh will have alternating major differences in conductance, there will in essence be dielectric capacitance coupling with high and low conductance areas alternating—thus, the mesh will serve as a series of anodes and alternating cathodes that would allow the electroporation energy to be delivered between one to the other); other bipolar iterations would be small eyelet patches where current will travel from one eyelet to the other on the ipsilateral or contralateral side; and yet another would be a bipolar design where a small clamp-like device is used with one end in the buckle mucosa of the mouth and the other on the cheek (this would have the simultaneous benefit of improving mucosal hygiene and cancer risk along with the cosmetic benefits to the face). This can also be advantageous for nighttime use of the device, in order to provide secure positioning of the device.

In some implementations of device 100, a suction device is included (e.g., a porous patch attached to a small suction device that is battery powered and serves as the power unit for electroporation as well). Here, by gently suctioning small amounts of the epidermis and underlying dermis, point electrodes are created that can serve as either composite or alternating bipoles. Specifically, with all the above bipolar designs, a phasic bipolar energy delivery system where the anode and cathode rapidly alternate is also envisioned. This is advantageous in some implementations because directional effects are undesired, i.e., akin to Botox injections, that tend to have concentrated effects at the site of injection.

In some implementations, another concept would involve capacitive touch technology both to deliver electroporation by creating an electrical field and for exact localization of where the contact is being made so as to touch trigger the dose of electroporation energy customized for that particular area. The present most commonly-used type of touchscreen technology involves capacitive localization. The screen itself is an insulator, there are underlying electrodes, and a user's finger serves as the second conductor, thus creating a capacitor. When touch occurs or with the degree of touch being made, a resulting quantifiable amount of current flow created by the electrical tension within the capacitor helps with the localization and commands that we can do when we touch the screen. The aspect here is the patient's skin serves as one conductor, and then a gel that is spiked with graphite particles, gold particles, or other similar particles along with a botulinum or other emollient containing gel can also be used. Then, when the smartphone or delivery device touches the skin, we will have a capacitor that's multifunctional. It will be multifunctional in that: a) because of the touch created, we will know exactly where on the skin we are, b) the deformation of the capacitor itself will serve as an energy delivery device without the need potentially for a battery, c) the electroporation thus generated will help disseminate the desired pharmaceutical agent in the gel into the skin, and d) the degree of touch can trigger differentiable commands in terms of the algorithm for delivery and the amount of delivery based on the perceived current.

Additional feedback algorithms are envisioned. Some implementations will use the electroporation signal as one or more test pulses for feedback changes and conductance of the skin, twitch response, piloerection, and other parameters. This is an advantageous feature since this method can encourage real-time feedback changes with high temporal resolution. Photographic parameters from the smartphone described above could also be used in additional to or as an alternative to such a test pulse(s).

In some implementations, device 100 can be configured for internal use. For example, the use of suction, the virtual capacitively coupled bipolar grid, etc., would can be used as part of endoscopes for colonic and esophageal and other GI mucosa premalignant conditions. In some implementations, office-based procedures where a small interdermal needle is placed to serve as the return limb for the electrical field placed over the skin can be performed. In some such implementations, a sodium or calcium containing gel (specifically 3% or higher concentration in sodium and 2% or higher calcium chloride concentration) is used where electroporation is initially done in a monopolar fashion or any of the bipolar fashions mentioned above to drive in the ion to the intradermal space. Then, once this is accomplished, the ion itself serves as the return electrode giving transdermal bipolar electroporation. The same treatments as described above (but from inside out for internal use) could be performed for GI applications.

In some implementations, ECG monitoring is included. ECG detection algorithms can be used to shut off energy delivery whenever a tachycardia or wide complex arrhythmia is elicited, for safety as part of the feedback techniques.

Tissue specific responses can be considered also. Each tissue has its own response curve or characteristics in response to electroporation. For skin treatment, dermal, smooth muscle, skeletal muscle, teeth and enamel, and subcutaneous fat all will have their own specificity. This specificity may be duration of pulse, voltage, pulse frequency, biphasic vs. single phase, or other output characteristics. This may also include duration of therapy at a given time. Device 100 can thus receive and interpret feedback that is related to each of these separately, such as dermal conductance for the dermis, myoelectrograms for the smooth and skeletal muscles, and transcutaneous impedance for fat. Depending on which continues to need treatment (which of the layers), then the real-time customized delivery of the energy characteristics ideal for that layer will continue to be delivered.

In some implementations, the tissue specific responses will prove useful in order to eliminate/sterilize areas that have been contaminated and/or infected with bacteria, viruses, or fungi. These aspects would be of utility because of the natural protection of the skin is either not intact, not efficient as normal tissue in immunity and defense from microorganisms, or because of the presence of microbiologic biofilm production or deposition at the site, as well as deregulated and reduced blood flow which would impair healing. Sterility would promote healing as well as prevent or treat infections (including breakdown or elimination of biofilms) via alterations of energy delivery, monitoring, and feedback mechanisms.

In some implementations, device 100 can be configured for use as an electoporative toothbrush. One way to do bipolar skin and buccal mucosal modulation is to use a conductive cream on the face when a user brushes their teeth with an electroporation toothbrush. Skin treatment will be done concurrently when brushing the teeth, and the electroporation now monopolar for the teeth may help with plaque removal, gum health, biofilm destruction or degradation, and buckle mucosal monitoring for premalignant conditions, etc. The skin gel will not need to be attached as an electrode per se, but could be linked via Bluetooth (for example) to a common base module. However, the option of directly linking, i.e., a skin patch placed while brushing the teeth but linked physically with a wire is also envisioned. Similarly, brushing or internal application of gel to any other body orifice for malignancy monitoring or serving as a bipole can also be done. In addition, other regions would include the anal/rectal region, vaginal region, or penile region.

Generic drug depots as also envisioned as part of the devices and techniques described herein. For example, as part of this feedback-based skin treatment device 100, small depot bags between the delivery tool and skin can be placed overnight in patients. In such an implementation, the feedback delivery tool will not only help with skin applications but also create a method to seamlessly delivery drugs when a patient sleeps, for example. This may be particularly useful for diabetic therapy such as with an electronated form of insulin that could be created specific for this therapy or modify existing insulin preparations. Similarly, hypertension management is highly desirable to be continuous and could be done with such a tool, again seamlessly overnight when the patient sleeps where the bedding itself could serve as the return electrode. Similarly, drug deposition of heart failure medications such as diuretics can be delivered in a continuous manner overnight to prevent fluid buildup while lying recumbent, which also enhances delivery of the drug to the nephrons. Similarly, in some cases a depot of anti-cancer agents can also be delivered in a continuous manner which may offer unique combinations of anti-cancer regimens based on slower-time based delivery and leverage different compounds to be more localized and lower dose to achieve the desired effect. This can also be useful with the aspects within this document that lead to real-time physiologic monitoring of parameters to implement a feedback circuit for titration of drug delivery. This can also be extended for feedback circuits the device uses skin monitoring as mentioned above, from a night-night comparison to alter delivery based on the prior night's skin characteristics.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A skin treatment device comprising:
a housing;
a first electrode, at least a portion of the first electrode extending from the housing;
a second electrode, at least a portion of the second electrode extending from the housing;
an insulator coupled to the housing and interposed between the first electrode and the second electrode;
a camera coupled to the housing or the insulator; and
a processor configured to control the skin treatment device to deliver DC electroporation energy from the first electrode in response to identifying an image captured by the camera as a skin irregularity.

2. The skin treatment device of claim 1, further comprising a skin temperature sensor.

3. The skin treatment device of claim 1, further comprising a humidity sensor.

4. The skin treatment device of claim 1, further comprising an on-board power source for supplying the DC electroporation energy.

5. The skin treatment device of claim 1, further comprising a pair of galvanic skin resistance (GSR) electrodes for measuring conductance of skin.

6. The skin treatment device of claim 1, further comprising a skin-contact electrode embedded within the insulator or positioned on an opposite side of a skin-contacting side of the insulator.

7. The skin treatment device of claim 1, wherein the device is configured as a hand-held toothbrush, and wherein the hand-held toothbrush is configured to deliver DC electroporation.

8. A method for treating a patient, the method comprising:
positioning a skin treatment device in contact with skin of the patient, the skin treatment device comprising:
a housing;
a first electrode, at least a portion of the first electrode extending from the housing;
a second electrode, at least a portion of the second electrode extending from the housing;
an insulator coupled to the housing and interposed between the first electrode and the second electrode;
a camera coupled to the housing or the insulator; and
a processor configured to control the skin treatment device; and
delivering DC electroporation energy from the first electrode in response to identifying, by the processor, a first image captured by the camera as a skin irregularity.

9. The method of claim 8, wherein the skin irregularity is a wrinkle.

10. The method of claim 8, wherein the skin irregularity is a cancerous or pre-cancerous lesion.

11. The method of claim 8, wherein the skin irregularity is a stretch mark or striae.

12. The method of claim 8, wherein the skin irregularity is: (i) acne or (ii) oral cancer of buccal mucosa, gums, or tongue.

13. The method of claim 8, wherein the DC electroporation energy is delivered automatically in response to the processor identifying the image captured by the camera as the skin irregularity.

14. The method of claim 13, further comprising ceasing the DC electroporation energy delivery based on identification, by the processor in a second image captured by the camera, of a reddening of skin at or near the skin irregularity.

15. The method of claim 13, further comprising increasing an intensity of the DC electroporation energy delivery based on a humidity detected by a humidity sensor of the skin treatment device.

16. The method of claim 13, further comprising increasing an intensity of the DC electroporation energy delivery based on a skin conductance detected by a pair of galvanic skin resistance (GSR) electrodes of the skin treatment device.

17. The method of claim 13, wherein the intensity is based on camera based visualization of degree of infection.

18. The method of claim 8, wherein the delivering DC electroporation energy comprises:
emitting the DC electroporation energy from the first electrode;
passing of the DC electroporation energy through the skin of the patient; and
receiving of the DC electroporation energy by the second electrode.
